(19) United States
(12) Patent Application Publication
(10) Pub. No.: US 2005/0153006 A1
(43) Pub. Date: Jul. 14, 2005

(54) Herbal bidi/beedi

(76) Inventors: Rohit Prakash, Copley, OH (US); Nayana Prakash, Copley, OH (US)

Correspondence Address: ROHIT PRAKASH, M.D., 1510 SOUTH HAMETOWN ROAD, COPLEY, OH, 44321, US

(21) Appl. No.: 10/307,784

(22) Filed: Dec. 2, 2002

(51) Int. Cl. A61K 35/78; A24B 15/00

(52) U.S. Cl. 424/774; 131/352

(57) ABSTRACT

Pre-determined and definite amounts of Herbal ingredients that are in the form of powdered herbs are mixed thoroughly together in a mixer. Clarified butter in its liquid form is added in small pre-determined amount to the mixture that is stirred again. Using automatic machines this mixture, which is dry and combustible, is filled inside the Tendu leaves to which filter tip is attached. Individual Bidis are packed together in boxes to be sold in the market. Alternatively, these Tendu leaves could be hand-filled with the Herbal mixture that can be stored in pouches.

### FED SPONSORSHIP

None (not applicable)

### CROSS REFERENCE TO RELATED APPLICATIONS

None (not applicable)

### DRAWINGS

None

### BRIEF SUMMARY

They are Tendu leaves, with or without filter, that are filled with an Herbal mixture as detailed below. These are smoked in the usual way (as tobacco Bidis). These will be generated either on automatic machines or hand filled.

The Herbal Bidi product serves several different purposes such as:
1. De-addiction from tobacco cigarette/Bidi smoking.
2. It can help in the cure of many diseases through the use of Herbal ingredients.
3. Smoking Herbal Bidis is soothing to the throat, helps the voice and improves digestive processes.
4. Smoking Herbal Bidis imparts a good aroma to the surroundings and is elevating for the mind.
5. The Herbal ingredients present in the Bidis have useful properties as listed in the Herbal literature and pharmacopoelas. They are listed below. Smoking them imparts some of their useful properties to the smoker.
6. Smoking Herbal Bidis is absolutely safe and, in fact is beneflcial, for our health.

### DETAILED DESCRIPTION

1. The Herbal ingredients mentioned below will be mixed in different proportions by weight depending on the flavor desired resulting in multi-flavored Herbal Bidis.

2. All of the ingredients, except clarified butter, are in their dry powder form.

3. The final looks of the finished product will be the same as the existing tobacco Bidis on the market today, which include different sizes, lengths and colors.

4. Following Herbal ingredients will be used. Their uses according to Herbal literature are listed in front of their names:
a. Turmeric: Stomachic, tonic, blood-purifier, anti-septic, carminative, appetizer, anti-spasmodic, anti-inflammatory etc.
b. Clove: Stain removal from teeth, salivary stimulator, buccal deodorant, gingival protection, flavoring agent, etc.
c. Licorice: Useful in Asthma, Bronchitis, chronic cough, mouth ulcers, heartburn, esophageal-gastric-duodenal inflammation and ulceration, anti-rheumatic etc.
d. Gotu Kola: Revitalizes nerves and brain, strengthens memory and intelligence, improves concentration, tonic, useful in skin diseases, Asthma and Bronchitis etc.
e. Indian Gooseberry: Anabolic, anti-bacterial, resistance building, expectorant, carminative, anti-oxidative, anti-viral, anti-emetic, ant-atherosclerotic etc.
f. Ink nut: Laxative, Stomachic, tonic, alterative, adaptogenic, anti-stress, Hepato-protective etc.
g. Cardamom: Aromatic, flavoring agent, stimulant, breath sweetener, digestive, aphrodisiac, digestive, appetizer, anti-colic, anti-flatulent etc.
h. Ginger: Carminative, GI stimulant, anti-depressant, anti-narcotic, anti-histaminic etc.
i. Sandalwood: Cooling, diaphoretic, diuretic, expectorant, useful in concentrating mind, headaches, skin troubles, burns etc.
j. Rose: Flavoring agent, soothing to Pitta, balancing to all doshas (Vata, Pitta and Kapha) etc.
k. Cinnamon: Aromatic, astringent, GI stimulant, expectorant, carminative, anti-bacterial, anti-fungal, useful in neuralgia, rheumatism, toothaches etc.
i. Indian Bdellium: Anti-inflammatory, anti-septic, stomachic, carminative, appetizer, digestive, diaphoretic, expectorant, diuretic, rejuvenative, de-toxifying, useful as a gargle in chronic tonsillitis, pharyngitis etc.
m. Bishop's Weed: Anti-spasmodic, tonic, carminative, anti-microbial, improves body's resistance etc.
n. Holy Basil: Heart tonic, purifies and invigorates body and mind etc.
o. Fenugreek: High in protein, useful externally on boils, ulcers etc.
p. Bay Leaves: Aromatic, flavoring agent etc.
q. Clarified Butter (Ghee): Balances doshas, makes all sensory organs strong and healthy, useful in hair growth, improves complexion, corrects tremors etc.

1. Protection is sought for exclusive production and marketing of this product in the consumer market as no one else has ever conceived of this Idea before.

2. Changing the proportion of ingredients or addition to or subtraction of Herbs from the mixture does not cause any material change in the product and should not be granted patent protection to those other than inventors who attempt this.

3. Protection is sought for exclusive making of different flavors of this product depending on the proportion of the ingredients or addition of flavoring agents.

4. Protection is sought for both means of making this product—by machine or hand filled.
